(19) United States
(12) Patent Application Publication
(10) Pub. No.: US 2025/0270010 A1
(43) Pub. Date: Aug. 28, 2025

(54) WATER BOTTLE WITH CONSUMPTION TRACKING AND PH SENSING CAP

(71) Applicant: Generosity Water, Inc., Montebello, CA (US)

(72) Inventors: Jasmine Davenport, Los Angeles, CA (US); Brendan Lake, Venice, CA (US); Micah Cravalho, Los Angeles, CA (US); Ariel Vergez, Cleveland, OH (US); Monty Wright, Lake Arrowhead, CA (US)

(73) Assignee: Generosity Water, Inc., Montebello, CA (US)

(21) Appl. No.: 19/061,482

(22) Filed: Feb. 24, 2025

Related U.S. Application Data
(60) Provisional application No. 63/558,392, filed on Feb. 27, 2024.

(51) Int. Cl. B65D 51/24; A45F 3/00; A45F 3/18; A61B 5/00; A61B 5/0531; G01F 23/284; G01K 1/14; G01N 33/18

(52) U.S. Cl. CPC B65D 51/245; A45F 3/18; A61B 5/0531; A61B 5/6887; G01F 23/284; G01K 1/14; G01N 33/18; A45F 2003/003

(57) ABSTRACT

A smart water bottle includes a liquid level sensor operable to generate sensor data regarding the volume of water in the bottle and transmit the data to at least one external server or device. The liquid level sensor is integrated into the cap and is a time of flight sensor operable to determine the distance of the surface of the water from the cap in order to determine volume. The smart water bottle further includes one or more pH sensors and/or total dissolved solids (TDS) sensors integrated into the cap. Advantageously, the cap is able to be detached from the cradle and nested within a cradle to begin inductive charging in order to increase longevity of the device.

### CROSS-REFERENCE TO RELATED APPLICATIONS

This application is related to and claims priority from the following US patents and patent applications: this application claims priority from and the benefit of U.S. Provisional Patent Application No. 63/558,392, filed Feb. 27, 2024, which is incorporated herein by reference in its entirety.

### BACKGROUND OF THE INVENTION

### 1. Field of the Invention

The present invention relates to smart water bottles, and more specifically to water bottles with consumption volume and pH monitoring sensors in the cap.

### 2. Description of the Prior Art

It is generally known in the prior art to provide smart water bottles and mobile applications for tracking water consumption over the course of a day.

Prior art patent documents include the following:

U.S. Pat. No. 11,013,353 for Wireless drink container for monitoring hydration by inventors Hambrock et al., filed Nov. 8, 2018, and issued May 25, 2021, discloses a wireless drink container able to monitor a person's hydration and prompt him or her to drink more if appropriate. The drink containers as described herein can monitor liquid levels and communicate with external devices about the liquid levels and rate of consumption. One or more sensors in the drink container monitor the liquid level within the container. A processor coupled to the sensor(s) estimates how much liquid has been removed from the container from changes in the liquid level and transmits a signal representing the change in liquid level to a smartphone or other external device. It also triggers an audio or visual indicator, such as an LED, that prompts the user to drink more based on the user's estimated liquid consumption and on the user's liquid consumption goals, which may be based on the user's physiology, activity level, and location.

US Patent Pub. No. 2022/0218137 for Smart container with interactive, colored lights by inventors Iverson et al., filed Apr. 30, 2020, and published Jul. 14, 2022, discloses a smart container including a bottle or other container, a load cell, an accelerometer, a processor, and a colored light source. The container may be a liquid container, a pill container, or a food container. The colored light source may include one or more light-emitting diodes (LEDs) which can be programed to emit unique illumination patterns. The container may also include a speaker or motor to emit audio and vibrational notifications. The container provides a method of tracking consumption by a user of a substance held in a container.

U.S. Pat. No. 9,506,798 for Liquid consumption tracker by inventors Saltzgiver et al., filed Feb. 1, 2016 and issued Nov. 29, 2016, discloses a sensor operative to track changes in a liquid level of a hand-held liquid container, the sensor comprising: an ultrasonic liquid-level sensor configured to indicate a liquid level of a liquid in a hand-held liquid container, wherein the ultrasonic liquid-level sensor is capable of indicating the liquid level when the surface of the liquid is less than 20 centimeters away from the ultrasonic liquid-level sensor; a radio transmitter; and a processor configured to draw power from a portable power source, receive liquid-level indications from the ultrasonic liquid-level sensor, and cause the radio transmitter to transmit data indicative of changes in the liquid level.

U.S. Pat. No. 9,380,897 for Fluid consumption monitoring system by inventors Pfeiffer et al., filed Jan. 27, 2016 and issued Jul. 5, 2016, discloses an activity and volume sensing beverage container cap system for a beverage container including a cap that couples with the beverage container, a processor, a timer and at least one activity level sensor, such as an inclinometer, coupled with the processor. The activity level sensor detects container orientation. When the container is tilted, the timer measures an amount of time in an orientation. Based on the current and previous orientation and the amount of time the container is in different orientations, the processor determines an amount of time of activity of the user, and a level of activity of the user, such as number of steps or moves per time. The inclinometer is also used to determine the amount of volume dispensed from the container when tilted over a threshold indicative of drinking. Thus one inclinometer may be utilized as both to determine activity level and volume of fluid dispensed.

U.S. Pat. No. 10,981,769 for Portable system for dispensing controlled quantities of additives into a beverage by inventors Lyons et al., filed Oct. 27, 2019, and issued Apr. 20, 2021, discloses a portable, self-contained beverage apparatus including a container assembly having a known storage capacity for storing a consumable liquid, and a dispensing assembly disposed within the container assembly that dispenses variable, non-zero quantities of additives into the consumable liquid. The dispensing assembly includes multiple apertures structured and arranged to retain vessels containing the additives to be dispensed into the consumable liquid. The beverage apparatus also includes a level sensor disposed within the container assembly that determines a consumable liquid level of the consumable liquid stored in the container assembly. In certain embodiments, one or more positive displacement pumping mechanisms are configured to pump additive liquid from additive containers into a beverage chamber.

WIPO Patent Pub. No. 2022/033666 for Smart bottle by inventors Villanueva Lopez et al., filed Aug. 11, 2020 and published Feb. 17, 2022, discloses a method for monitoring the use of a drinking bottle as well as a system for carrying out the method. According to the method a drinking bottle is provided which is designed to detect on basis of sensor data of at least one sensor whether the dispensing of liquid from the drinking bottle caused by drinking and to distinguish it from dispensing processes which do not represent drinking processes. In order to do so the evaluation system collects sensor data of the at least one sensor. The evaluation system evaluates this sensor data for detecting whether the dispensing has been caused by drinking and in case the dispensing has been caused by a drinking process stores the number of drinking processes and/or the volume of the liquid dispensing caused by drinking.

U.S. Pat. No. 11,185,179 for Liquid consumption monitoring device by inventor Zimbelman, filed Apr. 21, 2020 and issued Nov. 30, 2021, discloses a liquid consumption monitoring device for monitoring and encouraging consumption of a liquid including a cup and a lid, which is selectively engageable to the cup to close a top thereof. The user is positioned to tilt the cup to dispense a liquid from the cup, through an opening positioned in the lid, into a mouth of the user. A flow meter engaged to the cup, proximate to the opening, measures a volume of the liquid passing through the opening. An interface is engaged to at least one of the cup and the lid and is operationally engaged to the flow meter. The interface receives a signal from the flow meter when the liquid passes through the opening. The interface selectively actuates at least one of a speaker and a bulb, upon receipt of the signal from the flow meter, to provide a sensory reward to the user.

US Patent Pub. No. 2022/0167129 for Electronically enabled drinking receptacles by inventor Yang, filed Nov. 24, 2021, and published May 26, 2022, discloses an electronically enabled drinking receptacle that has a liquid container, an electronic processor, an electronic drinking monitor, and a user-communicator. The drinking monitor can provide information to the electronic processor regarding an amount of liquid withdrawn from the liquid container over time and the electronic processor can be configured to actuate the user-communicator to communicate information to a user. The user-communicator can include one or more lights on the drinking receptacle, and the information communicated to the user can be configured to encourage the user to drink more liquid. The drinking receptacle can include an electronic communicator configured to communicate information between the liquid container and a separate electronic device, which can be remote from the drinking receptacle. The separate electronic device can be a mobile electronic device such as a wearable mobile electronic device.

U.S. Pat. No. 10,676,251 for Smart drink container by inventor Krafft, filed Oct. 27, 2016, and issued Jun. 9, 2020, discloses a container for storing a liquid for consumption. The container comprising temperature and volume sensors, processors, energy source and a communication device to transmit the recorded temperature and volume data to a remote human interface. The container may also provide for various notifications and alerts to the user if the data falls outside predetermined variable ranges.

U.S. Pat. No. 10,329,061 for System and methods for managing a container or its contents by inventors Dias et al., filed Nov. 6, 2014, and issued Jun. 25, 2019, discloses a retainer, a lid, and a sensor, where the sensor is configured to detect information about the retainer, the lid, or the contents in the retainer. The sensor also may be configured to communicate with an internal or external computer system, thereby facilitating showing the detected information as a representation via a display element. In certain embodiments, the system may include an action element such as an open/close lid opening assembly configured to permit automatically or manually opening or closing a drink aperture or another type of dispensing aperture.

### SUMMARY OF THE INVENTION

The present invention relates to smart water bottles, and more specifically to water bottles with consumption volume and pH monitoring sensors in the cap.

It is an object of this invention to provide a smart water bottle having integrated water consumption tracking and tracking of other qualities of the water, in order to provide real-time hydration and health data for the user.

In one embodiment, the present invention is directed to a water bottle, including a water bottle body, a water bottle cap configured to attach to a top of the water bottle body, a sensor cradle extending downwardly from a bottom surface of the water bottle cap, wherein the sensor cradle includes a plurality of prongs, a plurality of total dissolved solids (TDS) sensors embedded in or attached to tips of the plurality of prongs, and a time of flight sensor embedded in the sensor cradle and surrounded by the plurality of prongs.

In another embodiment, the present invention is directed to a water bottle cap, including a sensor cradle extending downwardly from a bottom surface of the water bottle cap, wherein the sensor cradle includes a plurality of prongs, a plurality of total dissolved solids (TDS) sensors embedded in or attached to tips of the plurality of prongs, a time of flight sensor embedded in the sensor cradle and surrounded by the plurality of prongs, and at least one threaded surface configured to matingly connect with at least one threaded surface of a water bottle body.

In yet another embodiment, the present invention is directed to a water bottle, including a water bottle body, a water bottle cap configured to attach to a top of the water bottle body, a water bottle base configured to attach to a bottom of the water bottle body, one or more total dissolved solids (TDS) sensors, one or more time of flight sensors, one or more pH sensors, and/or one or more temperature sensors included within the water bottle cap, one or more intra-device antennas positioned in the water bottle cap, one or more external network antennas positioned in the water bottle base, wherein the one or more intra-device antennas are configured to transmit sensor data generated by the one or more total dissolved solids (TDS) sensors, the one or more time of flight sensors, the one or more pH sensors, and/or the one or more temperature sensors to the water bottle base, and wherein the one or more external network antennas are configured to transmit the sensor data to one or more external devices and/or one or more external servers.

These and other aspects of the present invention will become apparent to those skilled in the art after a reading of the following description of the preferred embodiment when considered with the drawings, as they support the claimed invention.

### BRIEF DESCRIPTION OF THE DRAWINGS

FIG. 1A illustrates a perspective view of a bottle according to one embodiment of the present invention.

FIG. 1B illustrates a side view of a bottle according to one embodiment of the present invention.

FIG. 2A illustrates a bottom perspective view of an intelligent bottle cap according to one embodiment of the present invention.

FIG. 2B illustrates a side sectional view of an intelligent bottle cap according to one embodiment of the present invention.

FIG. 2C illustrates a bottom orthogonal view of an intelligent bottle cap according to one embodiment of the present invention.

FIG. 2D illustrates a side orthogonal view of an intelligent bottle cap according to one embodiment of the present invention.

FIG. 3 illustrates a side sectional view of an intelligent bottle cap connected to a water bottle according to one embodiment of the present invention.

FIG. 4 illustrates a perspective sectional view of a basic bottle cap according to one embodiment of the present invention.

FIG. 5 illustrates a perspective sectional view of an intelligent bottle cap according to one embodiment of the present invention.

FIG. 6 illustrates a perspective view of a bottle cap and a corresponding charging system according to one embodiment of the present invention.

FIG. 7 illustrates a conceptual diagram of the electronic components of a bottle cap according to one embodiment of the present invention.

FIG. 8 illustrates a conceptual diagram of an intelligent water bottle connected to a network according to one embodiment of the present invention.

FIG. 9 illustrates a perspective view of an intelligent bottle cap according to one embodiment of the present invention.

FIG. 10 is a schematic diagram of a system of the present invention.

### DETAILED DESCRIPTION

The present invention is generally directed to smart water bottles, and more specifically to water bottles with consumption volume and pH monitoring sensors in the cap.

In one embodiment, the present invention is directed to a water bottle, including a water bottle body, a water bottle cap configured to attach to a top of the water bottle body, a sensor cradle extending downwardly from a bottom surface of the water bottle cap, wherein the sensor cradle includes a plurality of prongs, a plurality of total dissolved solids (TDS) sensors embedded in or attached to tips of the plurality of prongs, and a time of flight sensor embedded in the sensor cradle and surrounded by the plurality of prongs.

In another embodiment, the present invention is directed to a water bottle cap, including a sensor cradle extending downwardly from a bottom surface of the water bottle cap, wherein the sensor cradle includes a plurality of prongs, a plurality of total dissolved solids (TDS) sensors embedded in or attached to tips of the plurality of prongs, a time of flight sensor embedded in the sensor cradle and surrounded by the plurality of prongs, and at least one threaded surface configured to matingly connect with at least one threaded surface of a water bottle body.

In yet another embodiment, the present invention is directed to a water bottle, including a water bottle body, a water bottle cap configured to attach to a top of the water bottle body, a water bottle base configured to attach to a bottom of the water bottle body, one or more total dissolved solids (TDS) sensors, one or more time of flight sensors, one or more pH sensors, and/or one or more temperature sensors included within the water bottle cap, one or more intra-device antennas positioned in the water bottle cap, one or more external network antennas positioned in the water bottle base, wherein the one or more intra-device antennas are configured to transmit sensor data generated by the one or more total dissolved solids (TDS) sensors, the one or more time of flight sensors, the one or more pH sensors, and/or the one or more temperature sensors to the water bottle base, and wherein the one or more external network antennas are configured to transmit the sensor data to one or more external devices and/or one or more external servers.

The utilization of data tracking in managing everyday life has had benefits particularly in the health industry. The integration of either sensors or self-input data into web or mobile applications has allowed for more comprehensive and accurate diet tracking, fitness routines, and sleep quality tracking in ways previously not possible. One area of health that holds particular importance is hydration, not only for those regularly engaged in strenuous workouts, but even for office workers who often forget or otherwise neglect to consume enough water to maintain a healthy lifestyle.

One approach to hydration manage is to use mobile applications for manual logging and for user notifications. For example, apps such as WATERLLAMA allow a user to log types of beverages being drunk and sends notifications based on physical qualities of the user, such as sex, weight, daily activity, and weather to determine how often the user needs to drink. Much of this functionality, namely manual tracking and using health and personal data to determine a routine are common among a variety of apps including HYDRO COACH, WATERMINDER, and MY WATER, among other applications. However, apps such as these are reliant on user participation and engagement in order to generate data. Even missing a single day, then, is able to damage the usefulness of a longer period of data, especially if missed days tend to be associated with days with specific compliance levels, such as days that tend to be missed being low compliance days, which skews the data. While for some users, missing data does not represent a serious issue, for those with known health issues or with strenuous exercise requirements, such skewed data is capable of having significant health implications.

A system integrating actual sensor data corresponding to water consumption from at least one source (e.g., a water bottle) is important for measuring actual compliance with a hydration routine, even on days when an individual does not have the time or otherwise does not remember to log water consumption. Some systems have attempted to integrate sensors and sensor data transmission into water bottles, such as U.S. Pat. No. 11,013,353. The '353 patent includes a liquid level sensor disposed within the cavity of the bottle and an accelerometer to determine when the container tips. However, the '353 patent extolls the benefits of the use of such sensors as capacitive sensor or Hall effect sensors for using less power than other sensors for the liquid level sensor. Capacitive fluid measurements and Hall effect sensors, however, require specific orientations of the fluid in the container and often fail to detect liquid levels when the levels are low in the container. Furthermore, rather than being integrated into the cap, these sensors require a long extension to extend into the chamber in order to operate, which both limits the volume of water contained and requires an overly bulky and more easily damageable mechanism.

While some patents such as U.S. Pat. No. 9,506,798 mention use of an ultrasonic liquid level sensor for use in a water bottle cap, but only for the purpose of detecting liquid less than 20 centimeters away from the sensor, these systems, as well as those describing other types of liquid level sensor, fail to describe a reasonable method to charge or otherwise power the sensor devices. Instead, these systems either assume low power draw after which the caps or entire bottles are to be discarded, or describe systems with replaceable batteries. Furthermore, these systems do not provide any method to test the pH or total dissolved solids (TDS) of the water within the bottle, and therefore fail to monitor important quantities of the water.

The present invention further provides useful information for hydration in particular, as it accounts for both the TDS and quantity of water consumed. Because TDS content impacts the actual hydration provided by the water, providing information regarding both the TDS and the quantity of water consumed provides more detailed information regarding the actual hydration of the user than is provided by the prior art.

Referring now to the drawings in general, the illustrations are for the purpose of describing one or more preferred embodiments of the invention and are not intended to limit the invention thereto.

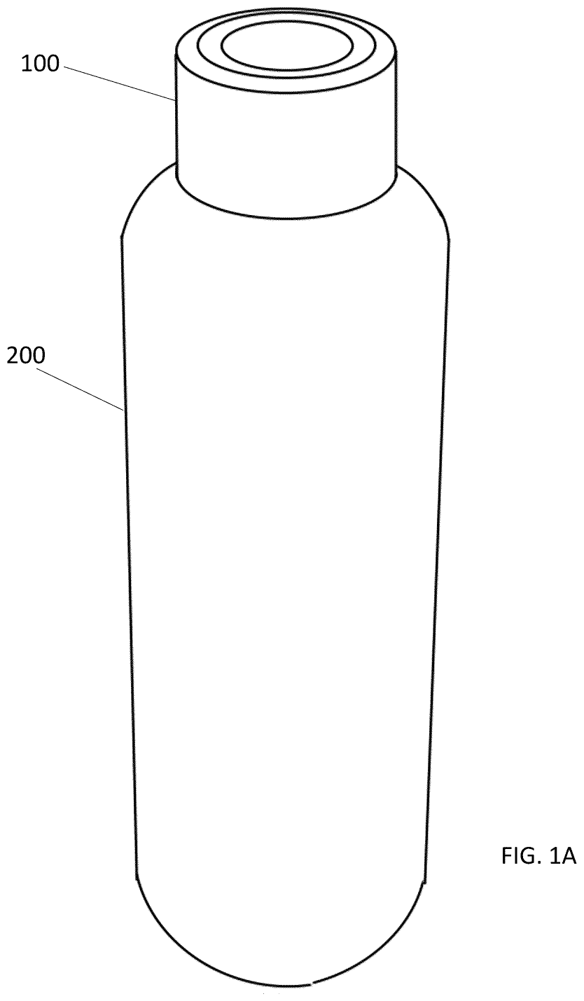

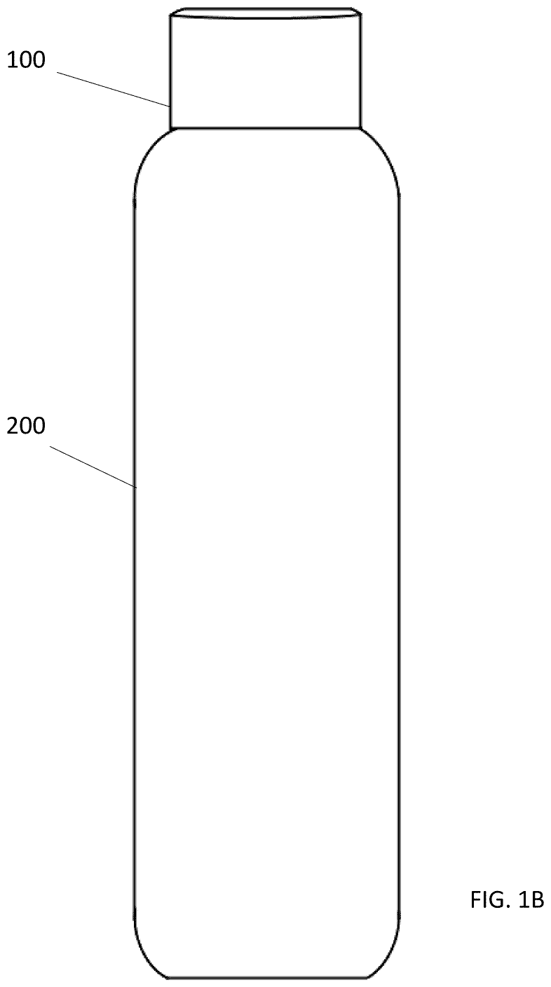

FIGS. 1A-1B illustrates a bottle according to one embodiment of the present invention. A water bottle includes a vessel 200 capped by a bottle cap 100 at a top end. In one embodiment, the vessel 200 is substantially cylindrical. In one embodiment, the bottle cap 100 is, similarly, substantially cylindrical. However, one of ordinary skill in the art will understand that other shapes for the water bottle (e.g., rectangular prismatic, etc.) are also contemplated and compatible with the present invention.

In one embodiment, the vessel 200 is formed from at least one metal material, including, but not limited to, stainless steel, aluminum, and/or at least one metal alloy. In one embodiment, the vessel 200 is formed from at least one composite material, including, but not limited to, carbon fiber and fiberglass. In one embodiment, the vessel 200 is formed from at least one hard polymer material, including polyethylene terephthalate (PET), polycarbonate (PC), high-density polyethylene (HDPE), and/or one or more other hard polymer materials. In one embodiment, the vessel 200 is formed from glass. In one embodiment, the inner surface and/or the out surface of the vessel 200 is coated with at least one powder coating and/or at least one silicone coating. In one embodiment, the vessel 200 includes a double wall design, but one of ordinary skill in the art will understand that other designs, including, but not limited to, single wall and triple wall configurations are also contemplated herein. In one embodiment, the materials and construction of the vessel 200 is configured to provide at least a 24-hour rating for cold fluids and/or at least a 12-hour rating for hot fluids. In one embodiment, the bottle is configured to hold approximately 1 liter, but one of ordinary skill in the art will understand that the bottle cap 100 of the present invention is operable to be affixed to different water bottles of various volumes. One of ordinary skill in the art will understand that the exterior of the bottle is able to include one or more shades of white, brown, red, pink, blue, black, and/or one or more shades of any other color, as the color is not intended to be limiting.

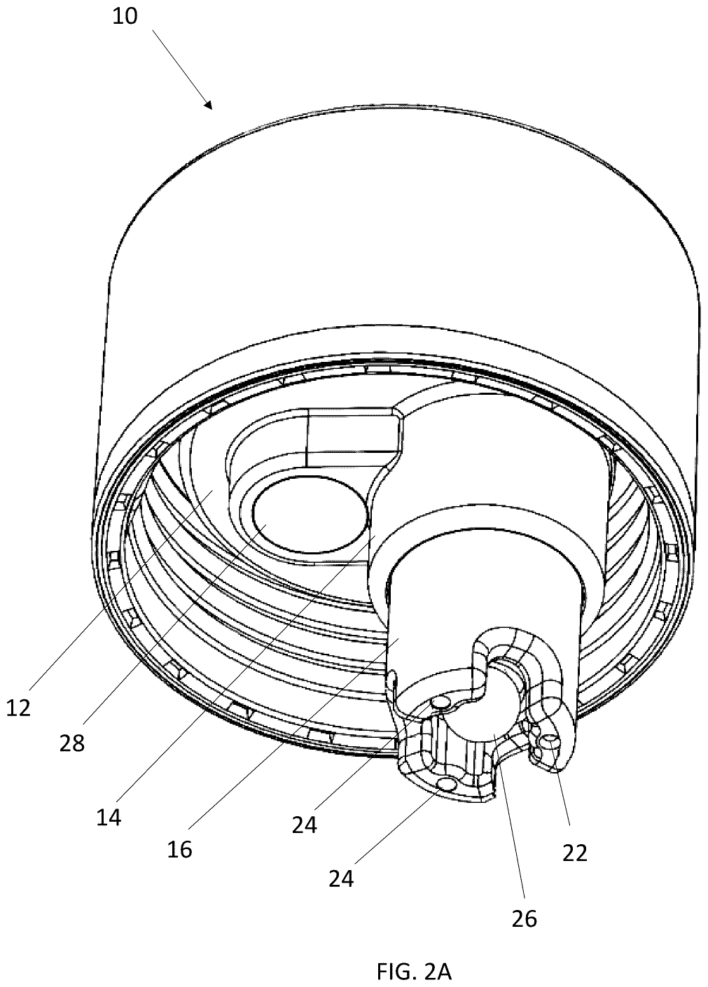

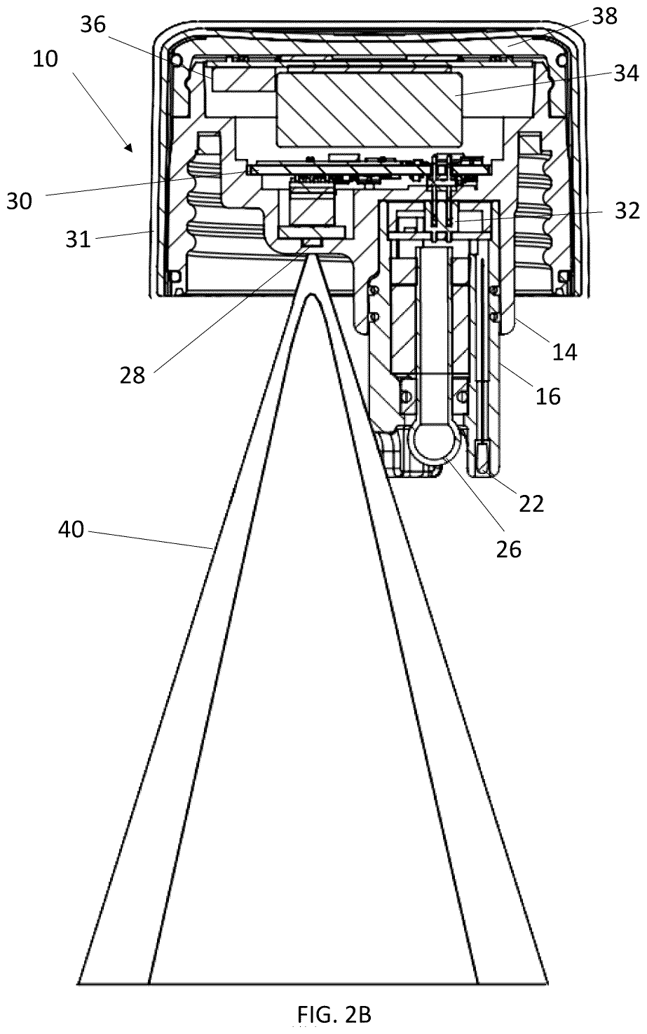

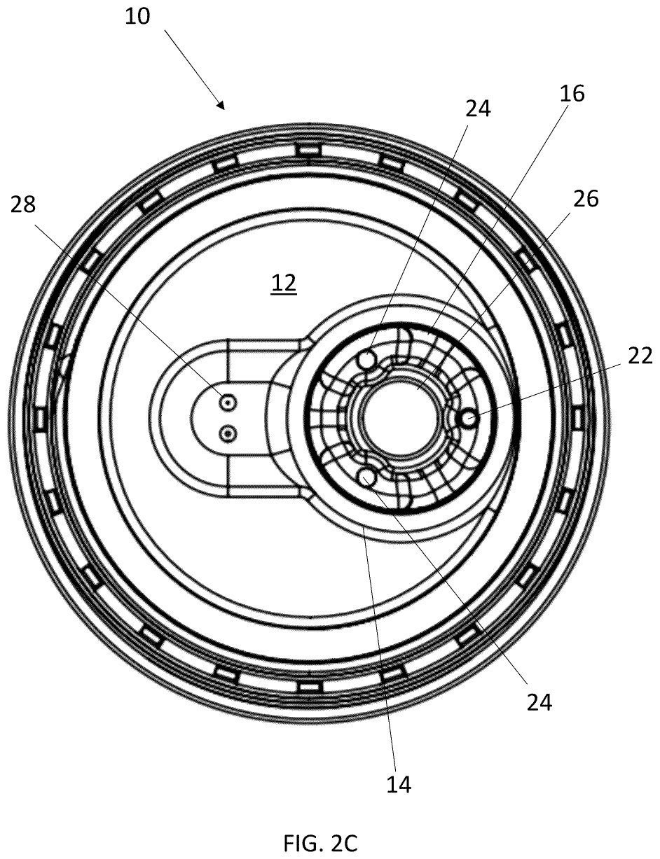

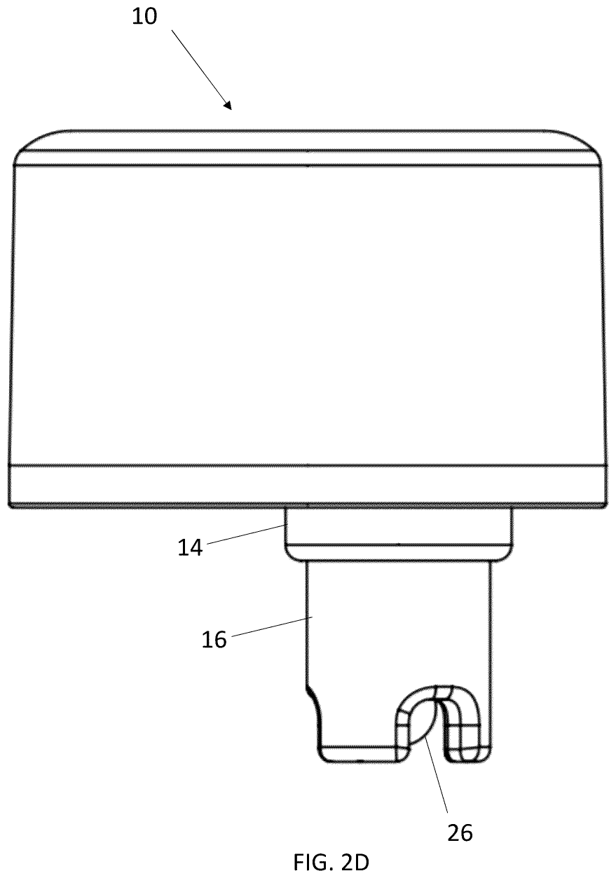

FIG. 2A illustrates a bottom perspective view of an intelligent bottle cap according to one embodiment of the present invention. The intelligent bottle cap 10 shown in FIG. 2A includes a bottom recession, including a plurality of threads lining an interior wall of the bottom recession, such that the bottle cap 10 is able to matingly fit with exterior threads on a top exterior surface of a water bottle. Alternatively, one of ordinary skill in the art will understand that the intelligent bottle cap 10 is instead able to include threads lining an exterior surface of the bottle cap 10 around the bottom recession, such that bottom of the bottle cap 10 is able to fit within an opening at the top of the water bottle and such that the threads lining the exterior surface of the bottle cap 10 matingly pair with threads lining an interior surface of the opening of the water bottle.

In a preferred embodiment, a sensor cradle 14 extends downwardly from a top surface 12 of a recession in the bottom of the bottle cap 10. In one embodiment, the sensor cradle 14 is configured to retain at least one time of flight sensor 28. In one embodiment, the at least one time of flight sensor 28 is aimed at and operable to detect time of flight (and correspondingly distances) along a longitudinal axis of the bottle cap 10 (i.e., toward the bottle to which the cap 10 is affixed). Therefore, the time of flight sensor 28 is operable to detect the distance between the bottle cap 10 and the surface of a fluid filling the bottle, thereby allowing the water level and volume of water within the bottle to be determined.

In one embodiment, the sensor cradle 14 also retains or is integrally formed with an extension 16 extending downwardly from the top surface 12 of the recession in the bottom of the bottle cap 10. A plurality of sensors are positioned at the bottom of the extension 16, and is configured to be of sufficient length such that extends beyond the bottom of the bottle cap 10. This allows the extension to be proximate to or positioned within the water or other liquid held in a bottle to which the bottle cap 10 is affixed. In one embodiment, one or more pH sensors 26 extends from approximately a center bottom section of the extension 16. In one embodiment, one or more TDS sensors 24 are attached to the bottom of the extension 16. In a preferred embodiment, two TDS sensors 24 are attached to the bottom of the extension 16. In one embodiment, one or more temperature sensors 22 are attached to the bottom of the extension 16. In one embodiment, as shown in FIG. 2A, three smaller prongs extend from an outer perimeter of a bottom end of the extension 16, two of which holds TDS sensors 24 and one of which holds a temperature sensor 22, with the three smaller prongs surrounding a central pH sensor 26. This three-prong configuration provides for improved protection of the central pH sensor 26 from, among other scenarios, bumping the extension 16 against the bottle or objects, and providing protection from debris coating the central pH sensor 26. The combination of both pH sensors and temperature sensors in the present invention is important for accurately determining the pH of the water, as the pH detected by the sensors is dependent on the temperature of the water. Therefore, in one embodiment, the bottle cap 10 includes a processor operable to receive data from both the central pH sensor 26 and the temperature sensor 22 and determine the actual pH of the water or other fluid in the bottle.

FIG. 2B illustrates a side sectional view of an intelligent bottle cap according to one embodiment of the present invention. As shown in FIG. 2B, the one or more pH sensors 26 are embedded in approximately the center of the extension 16, with a bulb of the one or more pH sensors 26 exposed at the bottom of the extension 16 in order to allow exposure to the water. Similarly, the temperature sensor 22 extends through an edge of the extension 16 such that it is exposed at the bottom.

The one or more pH sensors 26 and the other sensors within the extension 16 are connected to a central control board 30 of the bottle cap 10 via a plug 32 extending into an opening of the sensor cradle 14. In one embodiment, the extension 16 is easily detachable from the sensor cradle 14 via plugging and unplugging the extension 16 into the sensor cradle 14. This allows the extension 16, and the associated sensors within the extension 16, to be easily replaced or cleaned due to debris or other potential damage to the sensors in the extension 16.

The offset between the time of flight sensor 28 and the extension 16 is important. The cone of vision 40 of the time of flight sensor 28 is shown in FIG. 2B, with the offset of the extension 16 allowing the full cone of vision 40, or substantially the full cone of vision 40, to detect the distance to the water in the bottle. In one embodiment, the time of flight sensor 28 includes at least one lens configured to limit the cone of vision 40 of the time of flight sensor 28 to effectively focus the time of flight sensor 28 downward toward the surface of the water. In a preferred embodiment, the intelligent bottle cap 10 includes two time of flight sensors 28 directly adjacent to each other and each offset from the extension 16. As seen in FIG. 2B, the time of flight sensor 28 is also connected to the central control board 30.

In one embodiment, the bottle cap 10 includes a battery 34 positioned above the central control board 30. This is particularly important in embodiments where the bottle cap 10 is configured for wireless charging through a top end of the cap 10, as the battery 34 is positioned proximate to the top of the cap 10. In one embodiment, a speaker 36 (e.g., a piezo speaker) is positioned within the cap 10 proximate to the battery 34, near the top of the cap 10, as shown in FIG. 2B. In one embodiment, the speaker 36 is able to provide auditory indications of a state of the cap 10 (e.g., battery state, connectivity state, etc.) or of the water within the bottle (e.g., a quality of water, a volume of water, etc.).

In one embodiment, an upper shell 38 of the bottle cap 10 is formed from a non-metal material and/or a material that allows for the transmission of BLUETOOTH signals (or other wireless network signals) into and out of the bottle cap 10. This is important, as it allows for the transmission of commands into the cap 10 and/or the transmission of information out of the cap 10. In one embodiment, the upper shell 38 is substantially translucent or transparent, allowing light emitting diodes (LEDs) within the cap 10 to shine through the top of the cap 10. These LEDs are able to provide indications of a state of the cap 10 (e.g., battery state, connectivity state, etc.) and/or of the water within the bottle to which the cap 10 is attached (e.g., a quality of water, a volume of water, etc.).

Additional views of the cap 10 are able to be seen in FIGS. 2C and 2D.

In one embodiment, the cap 10 is overmolded with silicone 31 or another similar material. The overmolded silicone 31 provides an additional dust seal between the lid and the bottle when the lid is screwed on.

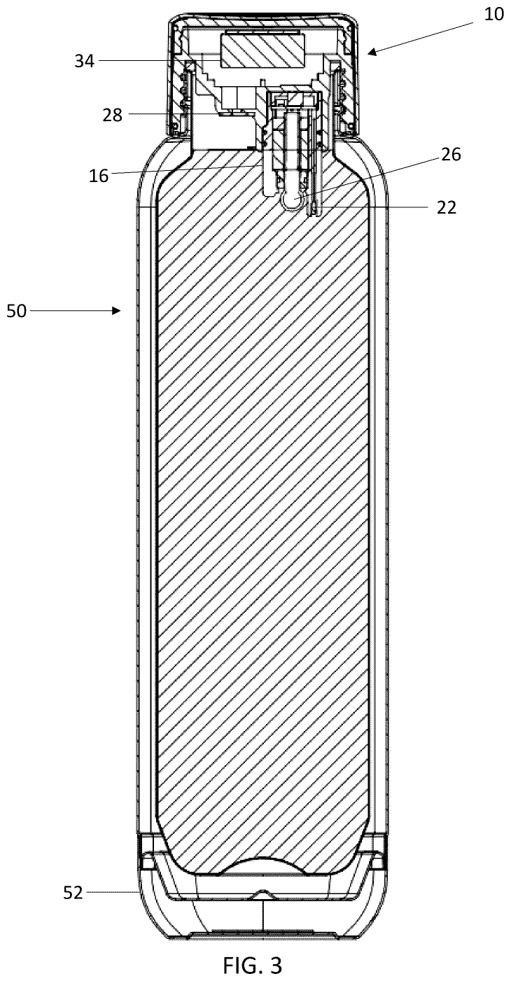

FIG. 3 illustrates a side sectional view of an intelligent bottle cap connected to a water bottle according to one embodiment of the present invention. As shown in FIG. 3, when the intelligent bottle cap 10 is attached to a bottle 50 and the bottle 50 is substantially full of water or other liquid, both the pH sensor 26 and the temperature sensor 22 extend into the water such that quality data is able to be generated. Additionally, though it is not shown in FIG. 3, the TDS sensor also ideally extends into the water or other liquid when in this state.

In one embodiment, the bottom or base portion 52 of the bottle 50 is an integral portion of the bottle 50 and does not include any electronic components. In another embodiment, the bottom or base portion 52 of the bottle 50 is removable (e.g., via unscrewed a threaded connection, undoing a latch, etc.). In one embodiment, the base portion 52 of the bottle 50 contains one or more “passive” electronic components (e.g., one or more identifier tags, such as RFID or NFC, or one or more LEDs indicating a status of the water and/or a status of the cap 10).

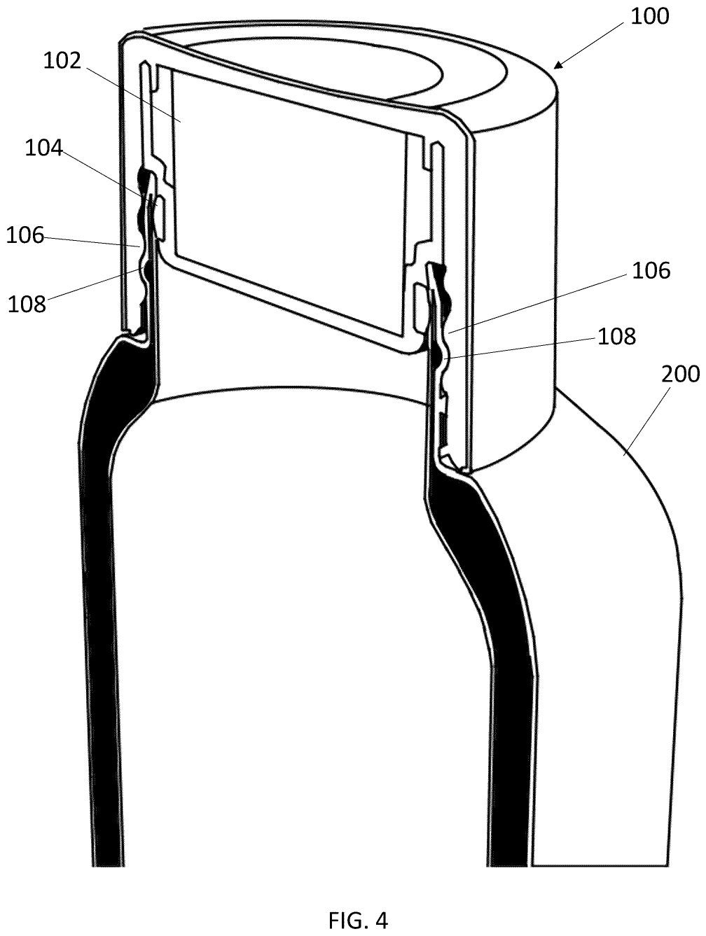

FIG. 4 illustrates a perspective sectional view of a bottle cap according to one embodiment of the present invention. In one embodiment, the bottle cap 100 includes a central stopper 102 operable to pug an opening in the top of the vessel 200. The bottle cap 100 includes cylindrical side walls extending downwardly from a top surface. In one embodiment, an inner surface of the cylindrical side walls includes a plurality of threads 106 configured to matingly engage with threads 108 positionally on the external sides of the top of the vessel 200. In one embodiment, the threads 108 are helical threads and/or annular threads. The threads allow the cap 100 to screw on the top of the vessel 200. In one embodiment, the stopper 102 includes a deformable sealing element 104 (e.g., an O-ring) surrounding an external side of a bottom portion of the stopper 102. In one embodiment, there is a small gap between the stopper 102 and the threads 106 of the internal side walls of the bottle cap 100. In one embodiment, the stopper 102 is a uniform rubber element, while, in another embodiment, the stopper includes electronic components for operating, processing, and/or powering one or more sensors. In one embodiment, one or more sensors are attached to or integrated into the bottom of the stopper 102.

In one embodiment, the one or more sensors include at last one ultrasonic transducer operable to transmit and receive ultrasonic waves in a pitch-catch orientation. The pitch-catch orientation allows distance to at least one feature (e.g., surface of a contained fluid) to be detected by time-of-flight analysis, with recognition of the fluid surface, as opposed to other features in the container being recognizable based on the impedance mismatch between the air and the fluid shown in the ultrasonic signal detected at a specific point in time. Distance between the ultrasonic transducer and the surface of the fluid allows the approximate volume of the fluid in the vessel 200 to be detected due to the known total volume of the vessel 200 and the distance to the fluid surface compared to the known height of the vessel 200 overall.

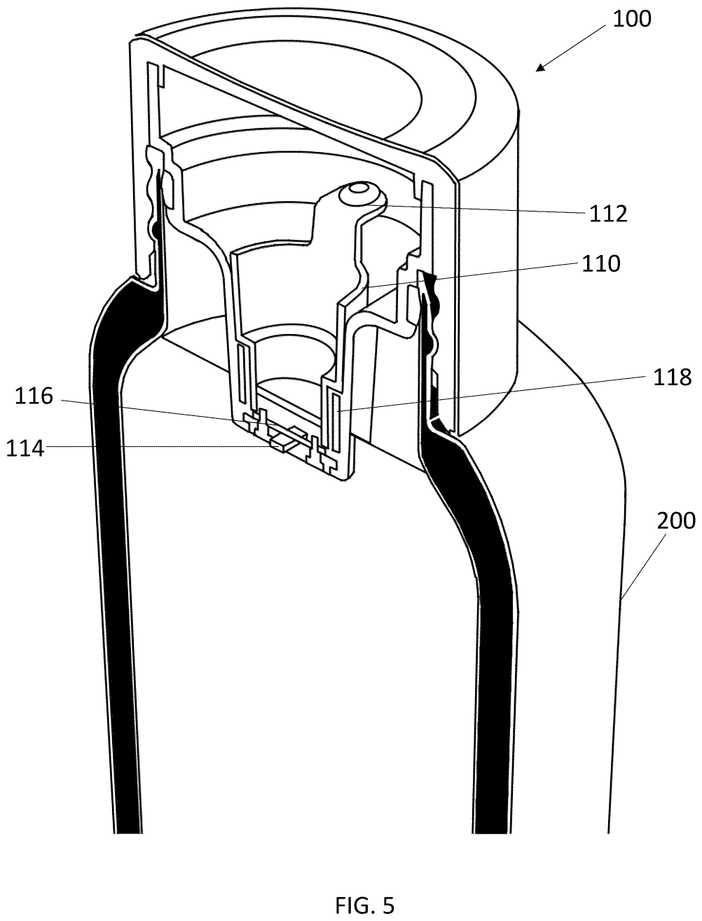

FIG. 5 illustrates a perspective sectional view of a bottle cap according to one embodiment of the present invention. In one embodiment, in addition to or in lieu of the stopper shown in FIG. 4, the bottle cap 100 includes a sensor cradle 110. In one embodiment, the sensor cradle 110 extends downwardly from a top of the bottle cap 100 and extends some length below the bottom of the bottle cap, ensuring that the bottom of the cradle 110 contacts the fluid in the vessel 200 when vessel 200 is substantially full. In one embodiment, the sensor cradle 110 is a tapered cylindrical extension. In one embodiment, the sensor cradle 110 or a portion of the sensor cradle 110 is secured to the internal side wall of the bottle cap 100 by at least one securing mechanism 112 (e.g., at least one bolt, at least one screw, by welding, etc.). In one embodiment, the bottom of the sensor cradle 110 includes at least one support 116 operable to hold at least one sensor 114. In one embodiment, the at least one sensor 114 includes at least one pH sensor, at least one TDS sensor, and/or at least one temperature sensor operable to determine one or more qualities of the fluid within. In one embodiment, at least one liquid level sensor is copositioned with the at least one sensor 114 and/or on a more raised portion of the sensor cradle 110 or interior surface of the bottle cap 100. In one embodiment, the sensor cradle 110 includes at least one visual or audio indicator 118. In one embodiment, the at least one visual or audio indicator includes at least one light emitting diode operable to activate or turn a specific color when the battery of the sensor cradle 110 is low, when abnormal fluid properties (e.g., too high or low pH) are present, and/or when disconnected to an external network. In one embodiment, a portion of the vessel 200 and/or the bottle cap 100 is transparent or translucent such that the at least one light emitting diode is able to be seen when the cap is attached. In one embodiment, the at least one visual or audio indicator includes at one speaker operable to play one or more audio alerts when the battery of the sensor cradle 110 is low, when abnormal fluid properties (e.g., too high or low pH) are present, and/or when disconnected to an external network.

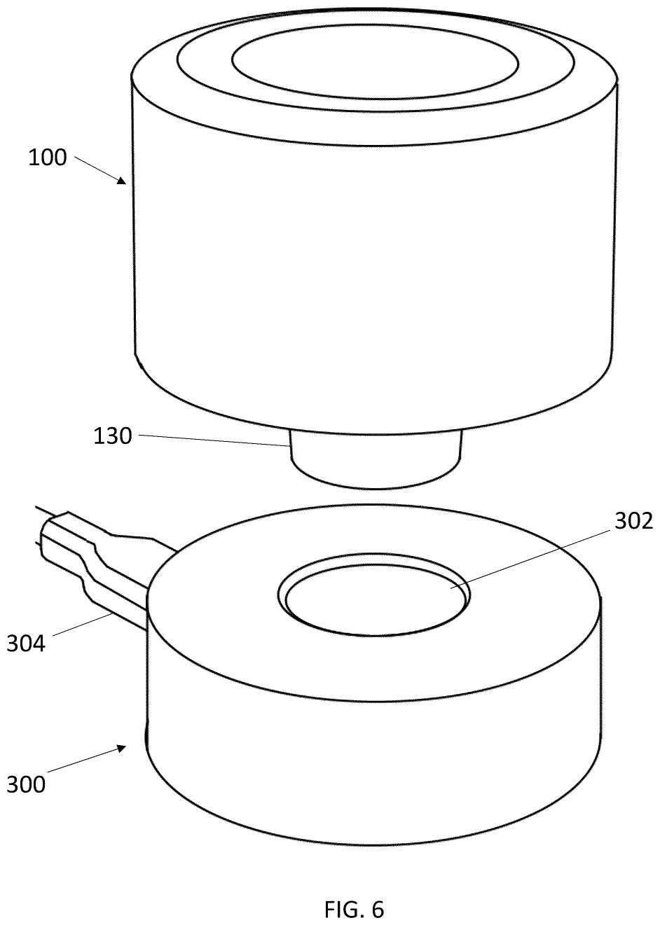

FIG. 6 illustrates a perspective view of a bottle cap and a corresponding charging system according to one embodiment of the present invention. In one embodiment, the bottle cap 100 includes an extension 130 extending downwardly from a bottom side of the bottle cap 100. In one embodiment, the extension 130 is the component that, when the bottle cap 100 is attached to the vessel, contains the sensors that detect quantities such as volume, pH, TDS, and others. In one embodiment, the extension 130 is sized and shaped to nest within a charging port 302 of a cradle 300.

In one embodiment, the cradle 300 is substantially cylindrically in shape. In one embodiment, the cradle 300 has substantially the same radius as the bottle cap 100, such that when the bottle cap 100 is nested in the cradle 300, the side walls of the cradle 300 are substantially flush with the side walls of the bottle cap 100, forming a clean, tower-like structure. In one embodiment, the cradle 300 is operable to be connected to at least one power source or at least one other external device via at least one cable 304. The at least one cable 304 is operable to be inserted into at least one power port of the cradle 300. One of ordinary skill in the art will understand that the type of port is able to be varied and includes USB-C, micro-USB, and/or any other type of cable port known in the art.

In one embodiment, the cradle 300 is operable to inductively charge the bottle cap 100 to fill the power storage used to power the components in the bottle cap 100. General methods for inductive charging are known in the art, and any such methods are compatible with the invention as recited herein. For example, in one embodiment, at least one coil is wrapped around and/or positioned beneath the charging port 302 of the cradle 300, where the at least one coil is operable to be energized with alternating current. In one embodiment, the alternating current is supplied by at least one internal power storage (e.g., a battery) within the cradle while, in another embodiment, the alternating current is supplied directly via the at least one cable 304 from at least one external power source. Other examples of inductive charging systems compatible with the present invention include, but are not limited to, those described in U.S. Pat. Nos. 11,569,685, 11,444,485, 11,398,747, 10,886,771, and 10,998,121, each of which is incorporated herein by reference in its entirety.

In one embodiment, additionally or alternatively to the use of inductive charging, the charging port 302 includes a plurality of electrical contacts positioned around the inner circumference of the charging port 302. In one embodiment, these contacts are configured to directly connect with and transmit power through one or more contacts positioned around the outer circumference of the extension 130 from the bottle cap 100. This provides a direct charging mechanism that provides for the same geometry as the inductively charging system.

In one embodiment, the cradle 300 includes at least one wireless antenna operable to communicate over a network with the bottle cap 100 and/or with at least one external server or device. In one embodiment, the cradle 300 is operable to transmit charging data to the at least one external server or device (e.g., an amount of power supplied over time, timestamp metadata for when power was supplied, magnitude of voltage drops, etc.). This data is able to be used to determine power consumption over long time periods and/or the current battery status of the bottle cap 100. In one embodiment, the cradle 300 is operable to transmit power storage data, indicating an amount of power still remaining in the cradle 300 (in embodiments where the cradle 300 includes internal power storage) to indicate when the cradle 300 needs to be connected to a power source.

In one embodiment, at least one pairing button extends outwardly from a top surface of the cradle 300 or from an external side wall of the cradle 300. In one embodiment, the at least one pairing button is flush with the surface of the cradle 300. Actuation of the at least one pairing button allows the cradle 300 to be “found” by a bottle cap 100 that is intended to be charged. In one embodiment, in order for the cradle 300 to be paired to the bottle cap 100, pairing buttons on both the cradle 300 and the bottle cap 100 need to be pressed simultaneously (or within a preset time, e.g., 1 second). In another embodiment, only buttons of the cradle 300 or the bottle cap 100 need to be pressed to pair.

In another embodiment, orientation of the bottle cap is configured such that the cradle 300 charges the bottle cap 100 from the top of the bottle cap 100, rather than the bottom. This is useful in embodiments, such as is shown in FIGS. 2A and 2B, where the battery is positioned toward the top of the cap 100 and the extension is not configured for charging. In one embodiment, a disk operable to be paired with the cradle 300 extends from a top of the bottle cap 100.

In one embodiment, the cradle 300 includes at least one LED operable to change colors to indicate a charging status of a bottle cap 100 (e.g., bottle cap is charging, bottle cap is connected but not charging, bottle cap is not connected, bottle cap is fully charged, etc.), a connection state of the cradle 300 to at least one external power source, a battery status of the cradle 300, a pairing state with at least one bottle cap 100, and/or other indications.

In one embodiment, the cradle 300 includes at least one biometric sensor. In one embodiment, the at least one biometric sensor includes at least one fingerprint scanner, at least one bioimpedance sensor, at least one retinal scanner, and/or any other suitable biometric sensor known in the art. In one embodiment, the cradle 300 only charges the bottle cap 100 if the cradle 300 detects one or more approved or known biometric signals via the at least one biometric sensor. In another embodiment, the at least one biometric sensor (e.g., the at least one bioimpedance sensor) is in the cap 100 or on a base of the bottle.

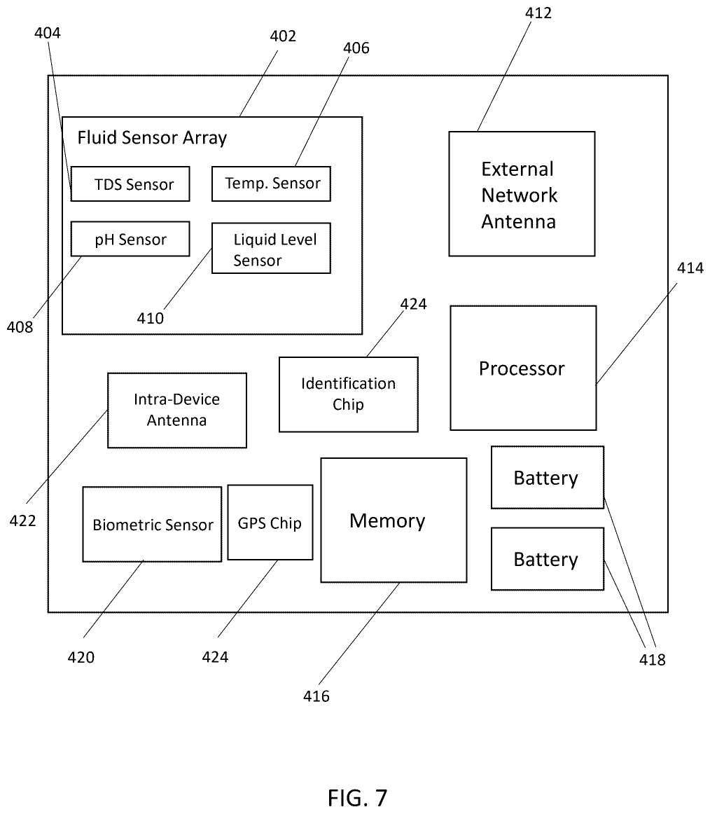

FIG. 7 illustrates a conceptual diagram of the electronic components of a bottle cap according to one embodiment of the present invention. One of ordinary skill in the art will understand that the conceptual diagram shown in FIG. 7 is not limiting as to the physical layout and orientation of the electric components of the bottle cap. The bottle cap includes a fluid sensor array 402 including one or more total dissolved solids (TDS) sensor 404, one or more temperature sensors 406, one or more pH sensors 408, and/or one or more liquid level sensors 410. The fluid sensor array 402 is configured to provide information regarding the fluid contained within the vessel.

In one embodiment, at least one external network antenna 412 is operable to transmit sensor data to at least one external server or device (e.g., smartphone, tablet, computer, smartwatch, etc.) via a network. In one embodiment, the network includes a wireless local area network (e.g., WI-FI), a wireless personal area network (e.g., BLUETOOTH), a cellular network, and/or any other type of wired or wireless network. In one embodiment the at least one external network antenna 412 is operable to receive commands to adjust one or more settings of the bottle cap and/or to receive one or more over-the-air updates to the firmware of the bottle cap.

In one embodiment, one or more sensors are positioned within a different part of the vessel (e.g., within a detachable base of the vessel) and are operable to transmit sensor data to and/or receive sensor data from the fluid sensor array 402 in the bottle cap via one or more intra-device antennas 422. In these embodiments, having sensors positioned in different locations allows sensors to be specially positioned to best provide data and prevents overcrowding of specific parts (e.g., the bottle cap), but the intra-device communication allows communication with an external device or server to go through a central point, simplifying the communications. For example, in one embodiment, the bottle cap transmits sensor data both from the fluid sensor array 402 and from other parts of the vessel (e.g., a detachable base) that is received through the intra-device antenna 422 via the external network antenna 412. In another embodiment, the external network antenna is instead positioned in another part of the device (e.g., a detachable base) that is operable to receive sensor data from the fluid sensor array 402 via the intra-device antenna 422 such that the other part of the device is the contact point with the external device or server.

In one embodiment, the cap includes a processor 414 and a memory 416. In one embodiment, the processor 414 is operable to determine basic functions, such as whether the TDS and/or pH of the liquid is above or below some minimum or maximum threshold ranges and transmitting signals to display visual or auditory warnings accordingly. The memory 416 is useful for temporarily storing sensor data, including timestamp metadata for the sensor data, before the sensor data is able to be transmitted by the external network antenna 412 to at least one external server or device (e.g., a smart phone, a tablet, a smart watch, a computer, etc.).

In one embodiment, the cap includes one or more batteries 418 operable to store charge and deliver power. In one embodiment, these batteries 418 are removable and replaceable with disposable or externally rechargeable batteries, but, in a preferred embodiment, the one or more batteries 418 act as a rechargeable power unit for the cap. One of the ordinary skill in the art will understand that the type (e.g., lithium ion, lead-acid, nickel-cadmium, lithium-sulfur, etc.), size, and/or number of batteries used in the present invention are not intended to be limiting.

In one embodiment, the cap includes at least one Global Positioning System (GPS) chip 424 or any other form of geolocation identifying sensor. The at least one GPS chip is operable to generate location data for the bottle, and is operable to correlate consumption data with the geolocation data to determine where water is consumed the most and at what locations water consumption is lacking, providing for more specific insights to the user. In one embodiment, the geolocation data is transmitted to at least one external device or server.

In one embodiment, the cap includes at least one identification chip 424, operable to be detected by at least one external sensor. In one embodiment, the at least one identification chip 424 includes at least one radiofrequency identification (RFID) chip and/or at least one near field communication (NFC) communication chip. In one embodiment, the cradle operable to charge the bottle cap includes a sensor for detecting the at least one identification chip 424. In one embodiment, the cradle and/or the cap are operable to transmit signals to at least one external device or server when the cradle detects the at least one identification chip 424, allowing for data to be produced regarding when charging begins and ends. In one embodiment, the cradle only charges the cap if the cradle detects one or more specific identification chips, allowing cradles to be specifically mated with one or more caps, while not charging others. In another embodiment, the cradle does not specifically detect identification chips and is agnostic as to the identity of the cap being charged.

In one embodiment, the cap includes at least one biometric sensor 420. By including at least one biometric sensor 420, the cap is operable to detect, based on at least one unique detected biometric identifier which user is currently drinking from the bottle. This allows multiple users to share the same bottle, or allow the main user to give small amounts of the water to someone else, while maintaining accurate data for each user. In one embodiment, the cap automatically restricts water flow through the cap if the biometric sensor 420 does not detect one or more recognized or approved users, but in another embodiment, the cap does not restrict drinking access based on the biometric sensor 420 readings. In one embodiment, the biometric sensor 420 automatically transmits an alert to at least one user device upon detection of a biometric reading that does not match one or more approved or recognized users of the bottle. In one embodiment, the biometric sensor 420 automatically transmits location data generated by the at least one GPS chip 424 if an unauthorized user is detected. In one embodiment, the at least one biometric sensor 420 includes at least one bioelectrical impedance sensor. The at least one bioelectrical impedance sensor allows, if the cap or one or more contacts extending out from the cap (e.g., adhering to an external side wall of the vessel) are contacted by a user at two points, the sensor is able to transmit a small alternating current through the body of the user and detect the returning signal to detect a unique impedance value for the user. Examples of bioimpedance sensor technology able to be included in the present invention include, but are not limited to, those described in U.S. Pat. Nos. 8,406,865 and 10,687,730 and U.S. Patent Pub. No. 2015/0157219, each of which is incorporated herein by reference in its entirety. In one embodiment, the biometric sensor 420 includes at least one fingerprint sensor, at least one retinal sensor, at least one voice detector, and/or any other form of biometric sensor known in the art.

In a preferred embodiment, the one or more pH sensors used in the present invention include at least one glass probe sensor (e.g., an agile glass probe). However, in another embodiment, the one or more pH sensors include one or more optical sensors and/or one or more other types of pH electrode. The present invention is advantageous over the prior art, as it utilizes a sufficiently small pH sensor to be used in a bottle cap, and specifically in the extension of the bottle cap. For example, in one embodiment, the bulb of the pH sensor, such as the pH sensor shown in FIGS. 2A and 2B, is approximately 7 mm in diameter.

In a preferred embodiment, the one or more TDS sensors used in the present invention include at least one conductivity sensor. In one embodiment, the one or more TDS sensors are replaced by and/or supplemented by one or more specific analyte sensors (i.e., one or more sensors able to detect specific types of dissolved materials in the water). In one embodiment, the one or more specific analyte sensors include one or more hyperspectral sensors. In one embodiment, the one or more specific analyte sensors are able to detect the individual levels of sodium (or sodium-based salts), potassium (or potassium-based salts), calcium (or calcium-based salts), magnesium (or magnesium-based salts), and/or other types of salts or ions. In one embodiment, the one or more specific analyte sensors are able to detect the individual levels of different metals, minerals, or microplastics within the water.

In a preferred embodiment, the one or more time of flight sensors used in the present invention include at least one laser or photon-emitting time of flight sensor (e.g., formed from a single-photon avalanche diode (SPAD)). In one embodiment, the photon-emitting time of flight sensor includes a plurality of diodes (e.g., a plurality of SPADs). In another embodiment, the one or more time of flight sensors include one or more ultrasonic time of flight sensors (e.g., a piezoelectric transducer element configured to be innervated to vibrate and release ultrasonic waves and optionally able to receive ultrasonic waves reflected by the top of the water).

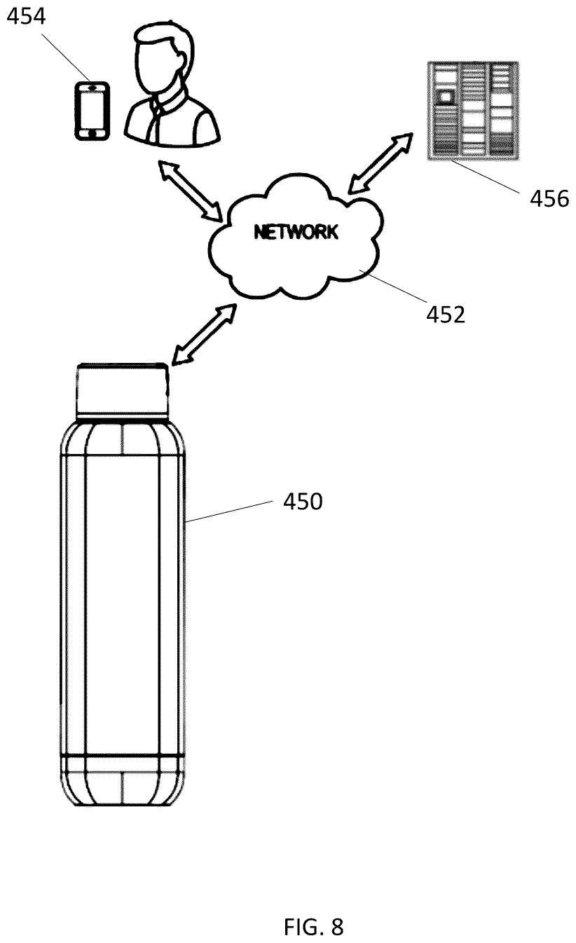

FIG. 8 illustrates a conceptual diagram of an intelligent water bottle connected to a network according to one embodiment of the present invention. In one embodiment, an intelligent water bottle 450 is connected to at least one network 452 (e.g., WI-FI network, BLUETOOTH network, cellular network, etc.). In one embodiment, at least one user device 454 is also connected to the at least one network 452 and operable to receive information concerning the intelligent water bottle 450 and/or operable to transmit commands to or about the intelligent water bottle 450. In one embodiment, the at least one network 452 includes at least one server and at least one database 456 for storing information received from the at least one user device 454 and/or the intelligent water bottle 450.

In one embodiment, data collected by the one or more pH sensors, the one or more TDS sensors, and/or the one or more temperature sensors are used to generate a water score, measuring the quality of the water within the bottle. In one embodiment, the water score is further based on data from one or more specific analyte sensors based on the presence or relative concentrations of one or more different analytes (e.g., different minerals, metals, salts, or microplastics). One of ordinary skill in the art will understand that the present invention is not intended to be limiting as to how the water score is presented. By way of example and not limitation, the water score is able to be presented as a number (out of 10, 100, etc.), a number of stars, a percentage value relative to an ideal, a series of different numbers measuring different sub-elements of the water score, or even a color on a spectrum (e.g., from red to violet). In one embodiment, a geolocation generated by a GPS chip, either on the intelligent bottle cap, or on a user device in network communication with the intelligent bottle cap, is transmitted to another user device or to one or more servers or databases. In one embodiment, the one or more servers or databases automatically correlates the geolocation with the water score. This allows the one or more servers or databases to build a state, national, global, or other geographical map of an area with associated water quality indicated for each area. In this way, the combination of the intelligent water bottle within the context of a larger network enables the development of a water quality equivalent of existing air quality maps. One of ordinary skill in the art will understand that geolocation detection is not to GPS tracking specifically, and is able to encompass other geolocating methods as well, including, but not limited to, triangulation via a plurality of WI-FI beacons, via IP geolocation, or via a cellular locator connected to the intelligent bottle cap or user device in network communication with the intelligent bottle cap, among other methods.

In one embodiment, an associated software application with the present invention is operable to transmit or generate an alert or notification on a user device when the user device enters into a geographic area with average water quality scores above or below preset thresholds.

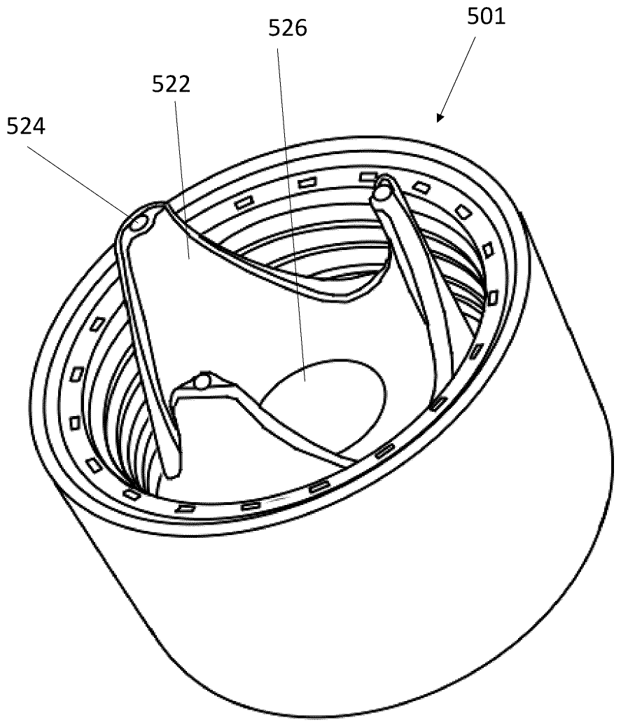

FIG. 9 illustrates a perspective view of an intelligent bottle cap according to one embodiment of the present invention. In one embodiment, an intelligent bottle cap 501 according to the present invention includes a central time of flight sensor 526 surrounded by a cradle 522 having a plurality of prongs (e.g., three prongs). Additionally or alternatively, the central pH sensor 526 is able to be replaced or supplemented by one or more pH sensors and/or temperature sensors used to determine the water level within the bottle at different times. At the end of each of the prongs 522 is at least one TDS sensor 524. The prongs 522 extend far enough from the base of the cap 501 such that the at least one TDS sensor 524 dips into the water within the bottle. In this embodiment, unlike the embodiment shown in FIG. 2A, the central time of flight sensor 526 is centered in the middle of the cap 501, with the cradle 522 spanning substantially the full inner surface area of the cap 501, providing for additional space for the central sensor 526 to operate. In one embodiment, one or more of the sensors 524 at the end of the prongs 522 is a temperature sensor.

Location data is created in the present invention using one or more hardware and/or software components. By way of example and not limitation, location data is created using the Global Positioning System (GPS), low energy BLUETOOTH based systems such as beacons, wireless networks such as WIFI, Radio Frequency (RF) including RF Identification (RFID), Near Field Communication (NFC), magnetic positioning, and/or cellular triangulation. By way of example, location data is determined via an Internet Protocol (IP) address of a device connected to a wireless network. A wireless router is also operable to determine identities of devices connected to the wireless network through the router, and thus is operable to determine the locations of these devices through their presence in the connection range of the wireless router.

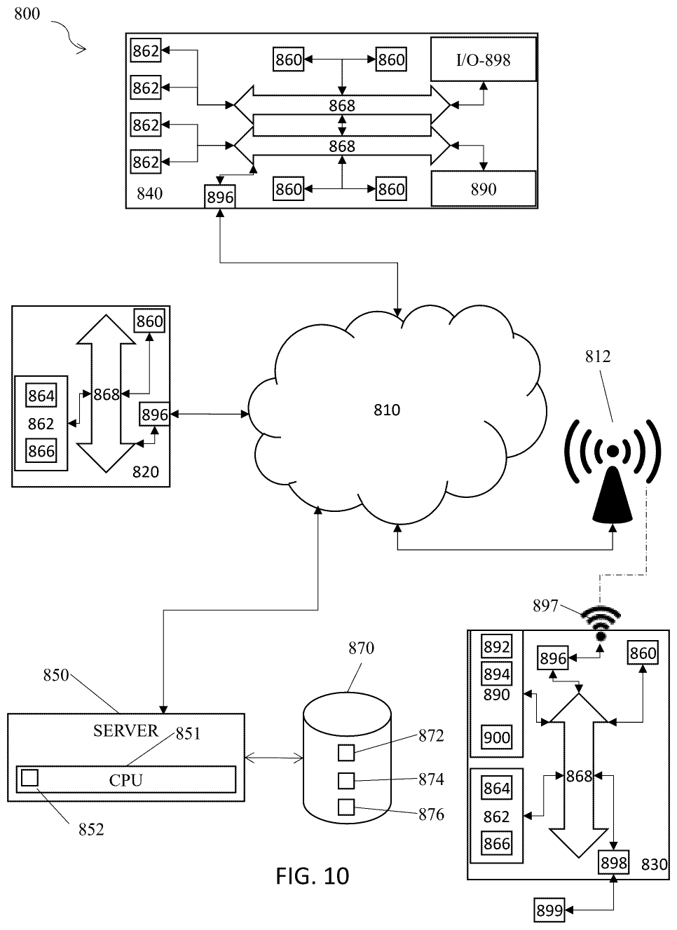

FIG. 10 is a schematic diagram of an embodiment of the invention illustrating a computer system, generally described as 800, having a network 810, a plurality of computing devices 820, 830, 840, a server 850, and a database 870.

The server 850 is constructed, configured, and coupled to enable communication over a network 810 with a plurality of computing devices 820, 830, 840. The server 850 includes a processing unit 851 with an operating system 852. The operating system 852 enables the server 850 to communicate through network 810 with the remote, distributed user devices. Database 870 is operable to house an operating system 872, memory 874, and programs 876.

In one embodiment of the invention, the system 800 includes a network 810 for distributed communication via a wireless communication antenna 812 and processing by at least one mobile communication computing device 830. Alternatively, wireless and wired communication and connectivity between devices and components described herein include wireless network communication such as WI-FI, WORLDWIDE INTEROPERABILITY FOR MICROWAVE ACCESS (WIMAX), Radio Frequency (RF) communication including RF identification (RFID), NEAR FIELD COMMUNICATION (NFC), BLUETOOTH including BLUETOOTH LOW ENERGY (BLE), ZIGBEE, Infrared (IR) communication, cellular communication, satellite communication, Universal Serial Bus (USB), Ethernet communications, communication via fiber-optic cables, coaxial cables, twisted pair cables, and/or any other type of wireless or wired communication. In another embodiment of the invention, the system 800 is a virtualized computing system capable of executing any or all aspects of software and/or application components presented herein on the computing devices 820, 830, 840. In certain aspects, the computer system 800 is operable to be implemented using hardware or a combination of software and hardware, either in a dedicated computing device, or integrated into another entity, or distributed across multiple entities or computing devices.

By way of example, and not limitation, the computing devices 820, 830, 840 are intended to represent various forms of electronic devices including at least a processor and a memory, such as a server, blade server, mainframe, mobile phone, personal digital assistant (PDA), smartphone, desktop computer, netbook computer, tablet computer, workstation, laptop, and other similar computing devices. The components shown here, their connections and relationships, and their functions, are meant to be exemplary only, and are not meant to limit implementations of the invention described and/or claimed in the present application.

In one embodiment, the computing device 820 includes components such as a processor 860, a system memory 862 having a random access memory (RAM) 864 and a read-only memory (ROM) 866, and a system bus 868 that couples the memory 862 to the processor 860. In another embodiment, the computing device 830 is operable to additionally include components such as a storage device 890 for storing the operating system 892 and one or more application programs 894, a network interface unit 896, and/or an input/output controller 898. Each of the components is operable to be coupled to each other through at least one bus 868. The input/output controller 898 is operable to receive and process input from, or provide output to, a number of other devices 899, including, but not limited to, alphanumeric input devices, mice, electronic styluses, display units, touch screens, gaming controllers, joy sticks, touch pads, signal generation devices (e.g., speakers), augmented reality/virtual reality (AR/VR) devices (e.g., AR/VR headsets), or printers.

By way of example, and not limitation, the processor 860 is operable to be a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and/or other manipulations of information.

In another implementation, shown as 840 in FIG. 10, multiple processors 860 and/or multiple buses 868 are operable to be used, as appropriate, along with multiple memories 862 of multiple types (e.g., a combination of a DSP and a microprocessor, a plurality of microprocessors, one or more microprocessors in conjunction with a DSP core).

Also, multiple computing devices are operable to be connected, with each device providing portions of the necessary operations (e.g., a server bank, a group of blade servers, or a multi-processor system). Alternatively, some steps or methods are operable to be performed by circuitry that is specific to a given function.

According to various embodiments, the computer system 800 is operable to operate in a networked environment using logical connections to local and/or remote computing devices 820, 830, 840 through a network 810. A computing device 830 is operable to connect to a network 810 through a network interface unit 896 connected to a bus 868. Computing devices are operable to communicate communication media through wired networks, direct-wired connections or wirelessly, such as acoustic, RF, or infrared, through an antenna 897 in communication with the network antenna 812 and the network interface unit 896, which are operable to include digital signal processing circuitry when necessary. The network interface unit 896 is operable to provide for communications under various modes or protocols.

In one or more exemplary aspects, the instructions are operable to be implemented in hardware, software, firmware, or any combinations thereof. A computer readable medium is operable to provide volatile or non-volatile storage for one or more sets of instructions, such as operating systems, data structures, program modules, applications, or other data embodying any one or more of the methodologies or functions described herein. The computer readable medium is operable to include the memory 862, the processor 860, and/or the storage media 890 and is operable to be a single medium or multiple media (e.g., a centralized or distributed computer system) that store the one or more sets of instructions 900. Non-transitory computer readable media includes all computer readable media, with the sole exception being a transitory, propagating signal per se. The instructions 900 are further operable to be transmitted or received over the network 810 via the network interface unit 896 as communication media, which is operable to include a modulated data signal such as a carrier wave or other transport mechanism and includes any delivery media. The term “modulated data signal” means a signal that has one or more of its characteristics changed or set in a manner as to encode information in the signal.

Storage devices 890 and memory 862 include, but are not limited to, volatile and non-volatile media such as cache, RAM, ROM, EPROM, EEPROM, FLASH memory, or other solid state memory technology; discs (e.g., digital versatile discs (DVD), HD-DVD, BLU-RAY, compact disc (CD), or CD-ROM) or other optical storage; magnetic cassettes, magnetic tape, magnetic disk storage, floppy disks, or other magnetic storage devices; or any other medium that can be used to store the computer readable instructions and which can be accessed by the computer system 800.

In one embodiment, the computer system 800 is within a cloud-based network. In one embodiment, the server 850 is a designated physical server for distributed computing devices 820, 830, and 840. In one embodiment, the server 850 is a cloud-based server platform. In one embodiment, the cloud-based server platform hosts serverless functions for distributed computing devices 820, 830, and 840.

In another embodiment, the computer system 800 is within an edge computing network. The server 850 is an edge server, and the database 870 is an edge database. The edge server 850 and the edge database 870 are part of an edge computing platform. In one embodiment, the edge server 850 and the edge database 870 are designated to distributed computing devices 820, 830, and 840. In one embodiment, the edge server 850 and the edge database 870 are not designated for distributed computing devices 820, 830, and 840. The distributed computing devices 820, 830, and 840 connect to an edge server in the edge computing network based on proximity, availability, latency, bandwidth, and/or other factors.

It is also contemplated that the computer system 800 is operable to not include all of the components shown in FIG. 10, is operable to include other components that are not explicitly shown in FIG. 10, or is operable to utilize an architecture completely different than that shown in FIG. 10. The various illustrative logical blocks, modules, elements, circuits, and algorithms described in connection with the embodiments disclosed herein are operable to be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application (e.g., arranged in a different order or partitioned in a different way), but such implementation decisions should not be interpreted as causing a departure from the scope of the present invention.

Certain modifications and improvements will occur to those skilled in the art upon a reading of the foregoing description. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the present invention.

The invention claimed is:

1. A water bottle, comprising:
a water bottle body;
a water bottle cap configured to attach to a top of the water bottle body;
a sensor cradle extending downwardly from a bottom surface of the water bottle cap, wherein the sensor cradle includes a plurality of prongs;
a plurality of total dissolved solids (TDS) sensors embedded in or attached to tips of the plurality of prongs; and
a time of flight sensor embedded in the sensor cradle and surrounded by the plurality of prongs.

2. The water bottle of claim 1, wherein the sensor cradle further includes one or more temperature sensors embedded in or attached to one or more of the tips of the plurality of prongs.

3. The water bottle of claim 1, wherein the time of flight sensor is positioned approximately in a center of the sensor cradle and approximately in a center of the water bottle cap.

4. The water bottle of claim 1, wherein a top surface of the water bottle cap includes a ring light emitting diode (LED) configured to indicate battery state, connectivity state, water volume, and/or water quality.

5. The water bottle of claim 1, wherein the water bottle cap includes one or more antennas configured to transmit sensor data from the plurality of TDS sensors and/or the time of flight sensor to at least one external device and/or at least one external server.

6. The water bottle of claim 1, further including a water bottle base configured to attach to a bottom of the water bottle body, wherein the water bottle base includes an external network antenna configured to transmit sensor data to at least one external device, and wherein the water bottle base is configured to receive the sensor data from an intra-device antenna located in the water bottle cap.

7. The water bottle of claim 1, wherein the water bottle cap includes one or more batteries.

8. The water bottle of claim 1, wherein the water bottle cap includes one or more geolocation sensors.

9. A water bottle cap, comprising:
a sensor cradle extending downwardly from a bottom surface of the water bottle cap, wherein the sensor cradle includes a plurality of prongs;
a plurality of total dissolved solids (TDS) sensors embedded in or attached to tips of the plurality of prongs;
a time of flight sensor embedded in the sensor cradle and surrounded by the plurality of prongs; and
wherein the water bottle cap is configured to attach to a top of a water bottle body.

10. The water bottle cap of claim 9, wherein the sensor cradle further includes one or more temperature sensors embedded in or attached to one or more of the tips of the plurality of prongs.

11. The water bottle cap of claim 9, wherein the time of flight sensor is positioned approximately in a center of the sensor cradle and approximately in a center of the water bottle cap.

12. The water bottle cap of claim 9, wherein a top surface of the water bottle cap includes a ring light emitting diode (LED) configured to indicate battery state, connectivity state, water volume, and/or water quality.

13. The water bottle cap of claim 9, wherein the water bottle cap includes one or more antennas configured to transmit sensor data from the plurality of TDS sensors and/or the time of flight sensor to at least one external device and/or at least one external server.

14. The water bottle cap of claim 9, wherein the water bottle cap includes at least one bioelectrical impedance sensor configured to generate biometric data identifying a user of the water bottle cap.

15. The water bottle cap of claim 9, wherein the water bottle cap further includes one or more batteries.

16. The water bottle cap of claim 9, wherein the water bottle cap further includes one or more geolocation sensors.

17. A water bottle, comprising:
a water bottle body;
a water bottle cap configured to attach to a top of the water bottle body;
a water bottle base configured to attach to a bottom of the water bottle body;
one or more total dissolved solids (TDS) sensors, one or more time of flight sensors, one or more pH sensors, and/or one or more temperature sensors included within the water bottle cap;
one or more intra-device antennas positioned in the water bottle cap;
one or more external network antennas positioned in the water bottle base;
wherein the one or more intra-device antennas are configured to transmit sensor data generated by the one or more total dissolved solids (TDS) sensors, the one or more time of flight sensors, the one or more pH sensors, and/or the one or more temperature sensors to the water bottle base; and
wherein the one or more external network antennas are configured to transmit the sensor data to one or more external devices and/or one or more external servers.

18. The water bottle of claim 17, wherein the water bottle cap includes at least one bioelectrical impedance sensor configured to generate biometric data identifying a user of the water bottle cap.

19. The water bottle of claim 17, wherein a top surface of the water bottle cap includes a ring light emitting diode (LED) configured to indicate battery state, connectivity state, water volume, and/or water quality.

20. The water bottle of claim 17, wherein the water bottle cap further includes one or more geolocation sensors.
